(19) United States
(12) Patent Application Publication
(10) Pub. No.: US 2008/0260691 A1
(43) Pub. Date: Oct. 23, 2008

(54) Prodrugs of Ribavirin with Improved Hepatic Delivery

(75) Inventors: Travis Mickle, Charlottesville, VA (US); Sven Guenther, Blacksburg, VA (US)

Correspondence Address: DARBY & DARBY P.C., P.O. BOX 770, Church Street Station, New York, NY, 10008-0770, US

(73) Assignee: New River Pharmaceuticals Inc., Radford, VA (US)

(21) Appl. No.: 11/718,311

(22) PCT Filed: Nov. 2, 2005
(86) PCT No.: PCT/US05/39621

§ 371 Date: Mar. 17, 2008

Related U.S. Application Data
(60) Provisional application No. 60/623,857, filed on Nov. 2, 2004.

(51) Int. Cl. A61K 38/16; C07K 2/00; A61P 31/12; A61K 38/21; C07K 9/00

(52) U.S. Cl. 424/85.7; 530/322; 514/8; 424/85.4

(57) ABSTRACT

The present invention relates ribavirin delivery systems and more specifically to compositions that comprise amino acids, as single amino acids or peptides, covalently attached to ribavarin and methods for administering conjugated ribavirin compositions.

### FIELD OF THE INVENTION

This invention relates to small molecule prodrugs of ribavirin that can be administered orally to preferentially deliver ribavirin to the liver. In particular, the invention includes the conjugation of ribavirin with small peptide or glycopeptide chains (2-5 amino acids with or without one carbohydrate moiety) primarily at, but not limited to, the 5′-position of the nucleoside analog. Generally, attachment of these peptides or glycopeptides may occur on any combination of one, two, or all three hydroxyl groups of the carbohydrate moiety of the nucleoside. This synthetic alteration may allow the new derivative to reversibly cross red blood cell membranes resulting in considerably reduced risk of hemolytic anemia. The conjugation may also promote selective delivery of the drug to the liver.

### BACKGROUND OF THE INVENTION

It is estimated that nearly 3% (˜200 million) of the world population and 1.8% (3.9 million) Americans have been infected with the hepatitis C virus (HCV). Between 75-85% of those infected develop a long-term infection, 70% suffer from chronic liver disease, 15% acquire cirrhosis, and approximately 3% die from the effects of the virus. Hepatitis is especially threatening for immunocompromised individuals, such as AIDS patients or organ recipients.

Ribavirin (FIG. 1) is an anti-viral drug that Schering-Plough Ltd. markets under license from Ribapharm as a therapy for the treatment of HCV infection. Ribavirin has also been found to be useful in the treatment of infant respiratory syncytial virus (RSV), influenza A (FLUAV) and B (FLUBV), hepatitis A (HAV) and B (HBV), Lassa fever virus (LFV), Hantaan virus (HTNV), and the respiratory virus that causes SARS. To date, the most effective treatment for HCV infection has been co-administration of the active agent ribavirin with interferon alpha-2a or b (IFN-α-2a or IFN-α-2b), or with peginterferon alpha-2a or b (PEG-IFN-α-2a or PEG-IFN-α-2b).

Ribavirin's mode of action is rather complex and not yet fully understood. It not only inhibits viral RNA and DNA chain replication but it also behaves as an immunomodulatory agent. Since the goal of antiviral treatment is to limit the infection of new cells to allow the immune system to eliminate infected cells, both modes of action give ribavirin a distinct advantage over other common anti-HCV agents.

While treatment with ribavirin is the most effective therapy for a number of viruses, it does have numerous drawbacks. The following warnings are listed for REBETOL® (oral ribavirin) indicating the range and severity of the toxicity of free ribavirin:

The primary toxicity of ribavirin is hemolytic anemia. The anemia associated with REBETOL therapy may result in worsening of cardiac disease that has lead to fatal and nonfatal myocardial infarctions. Patients with a history of significant or unstable cardiac disease should not be treated with REBETOL.

Significant teratogenic and/or embryocidal effects have been demonstrated in all animal species exposed to ribavirin. In addition, ribavirin has a multiple-dose half-life of 12 days, and so it may persist in nonplasma compartments for as long as 6 months. Therefore, REBETOL therapy is contraindicated in women who are pregnant and in the male partners of women who are pregnant. Extreme care must be taken to avoid pregnancy during therapy and for 6 months after completion of treatment in both female patients and in female partners of male patients who are taking REBETOL therapy. At least two reliable forms of effective contraception must be utilized during treatment and during the 6-month post-treatment follow-up period.

See Koren, G., et al., Can. Med. AsS. J. 2003, 168(10), 1289-1292.

Ribavirin exhibits significant toxicity, most commonly resulting in hemolytic anemia. The potential side effects in the current PEG-IFN-α and ribavirin therapies are a major area of concern for the medical community and explain why treatment is either discontinued or not administered. Individually both IFN-α and ribavirin have demonstrated gene toxicity and cytotoxicity along with consequent mild to serious side effects. Orally administered ribavirin has clearly shown dose-dependent hemolytic anemia and depression. The hemolytic anemia is due to the build-up of ribavirin 5′-triphosphate in red blood cells (RBC) and is reversible upon termination of treatment. This side effect limits the dose given to patients and may prevent further therapy with ribavirin in some individuals.

Pharmacologically, ribavirin has low and variable bioavailability (33-69%). This calls for the use of high dosages to obtain blood levels capable of inhibiting viral infection. The low bioavailability is caused by a large first-pass metabolic effect. A simplified metabolic pathway of orally administered ribavirin is outlined in FIG. 2. After reaching the gastrointestinal tract, a large portion of the drug (approx. 53%) is excreted in urine as intact drug or as metabolites (1,2,4-triazole-3-carboxamide and 1,2,4-triazole-3-carboxyclic acid) within 72-80 hours. Approximately 15% of a single oral dose is excreted in feces within 72 hours. The total bioavailibility of ribavirin averages 64%. From the plasma ribavirin is readily absorbed into different cell lines via nucleoside transporters where it is converted to the respective 5′-mono-, 5′-di-, and ultimately to the 5′-triphosphate by various kinases. This phosphorylation cascade is reversible in nucleated cells but irreversible in non-nucleated cells, such as red blood cells, which lack the phosphorylases required for the cleavage. For this reason, ribavirin accumulates in red blood cells causing hemolytic anemia.

Although ribavirin has been reported to also act on the immune system, in vitro studies demonstrated a synergistic effect with IFN-α inside infected cells. Miller, J. P., et al., J. Ann. N. Y. Acad. Sci. 1977, 284, 211-229; Weiss, R. C., et al., Veter. Microbiol. 1989, 20, 255-265. Moreover, an increase in iron levels in hepatocytes (associated with hemolysis) seems to diminish the efficacy of IFN-α. Okada, I., et. al, J. Lab. Clin. Med. 1992, 120, 569-723; Di Bisceglie, A. M., et al., J. Hepatol. 1994, 21, 1109-1112. Consequently, a reduction of hemolysis by use of a peptide/glycopeptide conjugate may enhance the long-term response to IFN-α during combination therapy. These results provide additional motivation for selective liver delivery.

Selective delivery of ribavirin to the liver should reduce the risks of the side effects associated with HCV therapy. However, selective drug delivery has always been a difficult hurdle to overcome. In the case of ribavirin, typical routes of selective therapy take advantage of prodrugs like viramidine (3-carboxamidine analog of ribavirin) that are converted in the liver to the parent compound (e.g., by adenosine deaminase for viramidine). Another common pathway of selective drug delivery uses macromolecules, in which the drugs are covalently attached to and eventually released from at the site of action. This form of transport usually relies on receptor binding of the macromolecule and in most cases, cannot be orally administered.

The present invention relates to small molecule prodrugs of ribavirin that can be administered orally to preferentially deliver ribavirin to the liver. In particular, we have produced a number of small peptide and glycopeptide conjugates of ribavirin. The potential use of peptide/glycopeptide derivatives of ribavirin represents a new approach of improving both major drawbacks of HCV therapy that have only been addressed individually in the past, namely significant reduction of side effects, and less invasive treatment method. Current prodrugs of ribavirin that show decreased toxicity require IV or IM administration. Free ribavirin can be taken orally (REBETOL®) but exhibits undesired side effects. Combining the advantages of both methods while eliminating, or at least considerably reducing their disadvantages by closely controlling the drug's pharmacokinetics demonstrate a novel strategy with significant therapeutic and commercial benefits.

For instance, the ribavirin peptide and glycopeptide conjugates of the present invention may serve to improve the toxicity profile of the parent drug. To reduce toxicity, the present invention attaches certain small peptides (2-5 amino acids) or glycopeptides (2-5 amino acids with 1-2 sugar moieties) to the carbohydrate moiety of ribavirin, which allows site-directed liver targeting and may prevent early degradation. Ribavirin peptide or glycopeptide derivatives (or “prodrugs”) that are stable to GI digestion but are primarily metabolized at the site of infection exhibit a significantly improved toxicological profile and enhanced bioavailability by circumventing the first-pass metabolism. Most of these ribavirin derivatives should be able to reversibly cross plasma cell membranes because their 5′—OH is blocked by a peptide and therefore cannot be phosphorylated until they are hydrolyzed in the liver (besides small amounts in other cells). As a result, no prodrug or its metabolites can accumulate in non-nucleated cells. This effect should considerably reduce the toxicity of the drug (especially hemolytic anemia) and may also improve selective delivery to the liver.

Another advantage of the ribavirin peptide and glycopeptide conjugates of the invention is the use of such conjugates as therapeutics for the treatment of antiviral diseases (e.g., HCV). Treatment could be simplified to exclusively oral administration making combination therapy (e.g., with interferon) redundant. Moreover, the coupling of ribavirin with small peptide or glycopeptide chains allows more variability and thus more possibilities for therapeutic optimization (e.g, reduced toxicity, increased bioavailability) compared to single amino acid derivatives. On the other hand, small peptide or glycopeptide derivatives are easier to prepare, characterize, and optimize than macromolecular (e.g., protein) derivatives of ribavirin.

### SUMMARY OF THE INVENTION

The present invention relates to the covalent attachment of ribavirin to a peptide or glycopeptide. The invention may be distinguished from the previous technologies by virtue of covalently attaching the ribavirin directly to the N-terminus, the C-terminus or to the side chain of an amino acid, an oligopeptide, a polypeptide (also referred to herein as a carrier peptide), or a glycopeptide.

In one embodiment, the invention provides a composition comprising a ribavirin covalently attached to an amino acid, a peptide, a dipeptide, a tripeptide, a polypeptide, or a glycopeptide. Preferably, the amino acid, dipeptide, polypeptide or glycopeptide comprise (i) one of the twenty naturally occurring amino acids (L or D isomers), or an isomer, analogue, or derivative thereof, (ii) two or more naturally occurring amino acids (L or D isomers), or an isomer, analogue, or derivative thereof, (iii) a synthetic amino acid, (iv) two or more synthetic amino acids or (v) one or more naturally occurring amino acids and one or more synthetic amino acids. Preferably, synthetic amino acids with alkyl side chains are selected from alkyls of C₁-C₁₇ in length and more preferably from C₁-C₆ in length. The peptide is preferably (i) an oligopeptide, (ii) a homopolymer of one of the twenty naturally occurring amino acids (L or D isomers), or an isomer, analogue, or derivative thereof, (iii) a heteropolymer of two or more naturally occurring amino acids (L or D isomers), or an isomer, analogue, or derivative thereof, (iv) a homopolymer of a synthetic amino acid, (v) a heteropolymer of two or more synthetic amino acids or (vi) a heteropolymer of one or more naturally occurring amino acids and one or more synthetic amino acids. The glycopeptide is preferably a peptide as described further defined by having attached thereto a carbohydrate.

In one embodiment, the ribavirin conjugate is attached to a single amino acid which is either naturally occurring or a synthetic amino acid. In another embodiment, the ribavirin conjugate is attached to a dipeptide, tripeptide, polypeptide or glycopeptide which could comprise any combination of the naturally occurring amino acids and synthetic amino acids. In another embodiment, the amino acids are selected from L-amino acids for digestion by proteases.

In another embodiment, the peptide carrier can be prepared using conventional techniques. A preferred technique is copolymerization of mixtures of amino acid N-carboxyanhydrides. In another embodiment, the peptide can be prepared through a fermentation process of recombinant microorganisms followed by harvesting and purification of the appropriate peptide. Alternatively, if a specific sequence of amino acids is desired, an automated peptide synthesizer can be used to produce a peptide with specific physicochemical properties for specific performance characteristics.

The ribavirin can be covalently attached to the side chains of the peptide or glycopeptide using conventional techniques. In a preferred embodiment a carboxylic acid containing ribavirin can be attached to the amine or alcohol group of the peptide side chain to form an amide or ester, respectively. In another embodiment, an amine containing ribavirin can be attached to the carboxylate, carbamide or guanine group of the side chain to form an amide or a new guanine group. In yet another embodiment, of the invention, linkers can be selected from the group of all chemical classes of compounds such that virtually any side chain of the peptide can be attached. In another embodiment, the ribavirin is directly attached to the amino acid without the use of a linker.

In another embodiment, direct attachment of a ribavirin to the carrier peptide or glycopeptide may not form a stable compound and therefore the incorporation of a linker between the ribavirin and the peptide is required. The linker should have a functional pendant group, such as a carboxylate, an alcohol, thiol, oxime, hydraxone, hydrazide, or an amine group, to covalently attach to the carrier peptide.

The invention also provides a method for preparing a composition comprising a ribavirin covalently attached to a peptide or glycopeptide. The method comprises the steps of:

(a) attaching the ribavirin to a side chain (and/or N-terminus and/or C-terminus) of an amino acid to form a ribavirin/amino acid complex;

(b) forming an amino acid complex N-carboxyanhydride (NCA) or forming a ribavirin/amino acid complex NCA from the ribavirin/amino acid complex; and

(c) polymerizing the ribavirin/amino acid complex N-carboxyanhydride (NCA).

Another embodiment, of the present invention is use of the ribavirin conjugates to provide dosage form reliability and batch-to-batch reproducibility.

In another embodiment, the invention provides a method for delivering ribavirin to a patient, the patient being a human or a non-human animal, comprising administering to the patient a composition comprising a ribavirin covalently attached to a peptide or glycopeptide. In one embodiment, the ribavirin is released from the composition by enzyme catalysis. In another embodiment, the ribavirin is released in a time-dependent manner based on the pharmacokinetics of the enzyme-catalyzed release.

The compositions of the invention can also include one or more microencapsulating agents, adjuvants and pharmaceutically acceptable excipients. The ribavirin can be bound to the microencapsulating agent, the adjuvant or the pharmaceutically acceptable excipient through covalent, ionic, hydrophilic interactions or by some other non-covalent means. The microencapsulating agent can be selected from polyethylene glycol (PEG), amino acids, carbohydrates or salts. In another embodiment, when an adjuvant is included in the composition, the adjuvant preferably imparts better absorption either through enhancing permeability of the intestinal or stomach membrane or activating an intestinal transporter.

It is another embodiment, of the present invention that the ribavirin may be combined with peptides of varying amino acid content to impart specific physicochemical properties to the conjugate including, molecular weight, size, functional groups, pH sensitivity, solubility, three dimensional structure and digestibility in order to provide desired performance characteristics.

In another preferred embodiment, the amino acid chain length can be varied to suit different delivery criteria. For delivery with increased bioavailability, the ribavirin may be attached to a single amino acid to eight amino acids, with the range of two to five amino acids being preferred. For modulated delivery or increased bioavailability of ribavirin, the preferred length of the oligopeptide is between two and 50 amino acids in length. In another embodiment, the carrier peptide controls the solubility of the ribavirin-peptide or ribavirin-glycopeptide conjugate and is not dependant on the solubility of the ribavirin. Therefore, the mechanism of sustained or zero-order kinetics afforded by the conjugate-ribavirin composition avoids irregularities of release and cumbersome formulations encountered with typical dissolution controlled sustained release methods.

In another embodiment, the ribavirin may be attached to an adjuvant recognized and taken up by an active transporter. In one embodiment, the active transporter is not the bile acid active transporter. In another embodiment, the invention does not require the attachment of the ribavirin to an adjuvant recognized and taken up by an active transporter for delivery.

In another embodiment, the carrier peptide allows for multiple ribavirins to be attached. The conjugates provide the added benefit of allowing multiple attachments of ribavirin moieties or other modified molecules which can further modify delivery, enhance release, targeted delivery, and/or enhance adsorption. In a further embodiment, the conjugates may also be combined with adjuvants or be microencapsulated.

In another embodiment, the conjugates provide for a wide range of pharmaceutical applications including drug delivery, cell targeting, and enhanced biological responsiveness.

In another preferred embodiment, the composition of the invention is in the form of an ingestible pill, tablet or capsule, an intravenous preparation, an intramuscular preparation, a subcutaneous preparation, a depot implant, a transdermal preparation, an oral suspension, a sublingual preparation, an intranasal preparation, inhalers, or anal suppositories. In another embodiment, the peptide or glycopeptide is capable of releasing the ribavirin from the composition in a pH-dependent manner.

In another embodiment, following administration of the ribavirin conjugate by a method other than oral, first pass metabolism is prevented, by avoiding recognition of liver oxidation enzymes due to its peptidic structure.

The invention also provides a method for controlling release of a ribavirin from a composition wherein the composition comprises a peptide or glycopeptide, and the method comprises covalently attaching the ribavirin to the peptide or glycopeptide. It is a further embodiment of the invention that enhancement of the performance of ribavirin from a variety of chemical and therapeutic classes is accomplished by extending periods of sustained blood levels within the therapeutic window. For a drug where the standard formulation produces good bioavailability, the serum levels may peak too fast and too quickly for optimal clinical effect as illustrated below. Designing and synthesizing a specific peptide conjugate that releases the ribavirin upon digestion by intestinal enzymes mediates the release and absorption profile thus maintaining a comparable area under the curve while smoothing out ribavirin absorption over time.

Conjugate prodrugs may afford sustained or extended release to the parent compound. Sustained release typically refers to shifting absorption toward slow first-order kinetics. Extended release typically refers to providing zero-order kinetics to the absorption of the compound. Bioavailability may also be affected by factors other than the absorption rate, such as first pass metabolism by the enterocytes and liver, and clearance rate by the kidneys. Mechanisms involving these factors require that the drug-conjugate is intact following absorption. The mechanism for timed release may be due to any or all of a number of factors. These factors include: 1) gradual enzymatic release of the parent drug by luminal digestive enzymes, 2) gradual release by surface associated enzymes of the intestinal mucosa, 3) gradual release by intacellular enzymes of the intestinal mucosal cells, 4) gradual release by serum enzymes, 5) conversion of a passive mechanism of absorption to an active mechanism of uptake, making drug absorption dependent on the Km for receptor binding as well as receptor density, 6) decreasing the solubility of the parent drug resulting in more gradual dissolution 7) an increase in solubility resulting in a larger amount of drug dissolved and therefore absorption over a longer period of time due to the increased amount available.

The potential advantages of enzyme mediated release technology extend beyond the examples described above. For instance ribavirin conjugate can benefit from increased absorption achieved by covalently bonding those ribavirin to one or more amino acids of a peptide and administering the drug to the patient as stated earlier. The invention also allows targeting to intestinal epithelial transport systems to facilitate absorption of ribavirins. Better bioavailability, in turn, may contribute to lower doses being needed. Thus it is a further embodiment of the invention that by modulating the release and improving the bioavailability of a ribavirin in the manner described herein, reduced toxicity of the ribavirin can be achieved.

In another embodiment of the present invention the amino acids used can make the conjugate more or less labile at certain pH's or temperatures depending on the delivery required. Further, one embodiment, the selection of the amino acids will depend on the physical properties desired. For instance, if an increase in bulk or lipophilicity is desired, then the carrier polypeptide will include glycine, alanine, valine, leucine, isoleucine, phenylalanine and tyrosine. Polar amino acids, on the other hand, can be selected to increase the hydrophilicity of the peptide. In another embodiment, the amino acids with reactive side chains (e.g., glutamine, asparagine, glutamic acid, lysine, aspartic acid, serine, threonine and cysteine) can be incorporated for multiple attachment points for ribavirin or adjuvants to the same carrier peptide.

In another embodiment, the invention provides methods of testing the conjugates using Caco-2 cells.

It is to be understood that both the foregoing general description and the following detailed description are exemplary, but not restrictive, of the invention. These and other aspects of the invention as well as various advantages and utilities will be more apparent with reference to the detailed description of the preferred embodiments and in the accompanying figures.

### BRIEF DESCRIPTION OF THE DRAWINGS

The invention is best understood from the following detailed description when read in connection with the accompanying figures.

FIG. 1 depicts the structure of ribavirin.

FIG. 2 illustrates a comparison of ribavirin metabolism (left) and potential metabolism of ribavirin peptide/glycopeptide conjugates (right).

FIG. 3 illustrates serum levels of AZT vs. two batches of Poly(Glu)-AZT as determined by ELISA analysis.

FIG. 4 illustrates a comparison of the levels of AZT/Glu(AZT)n detected by ELISA and the levels of free AZT detected by LC-MS/MS.

FIG. 5 depicts amino acid prodrugs of AZT.

FIG. 6 illustrates the amount of free drug detected by LC-MS/MS after IV administration of parent drug (red, squares) and pentapeptide prodrug (blue, diamonds).

FIG. 7 depicts the general structure of ribavirin dipeptides.

FIG. 8 depicts the structure of a glycopeptide derivative of ribavirin comprising a carbamate linkage.

FIG. 9 illustrates a sample scheme for the synthesis of ribavirin peptide derivatives with various chain lengths.

FIG. 10 illustrates a sample scheme for the synthesis of a ribavirin glycopeptide derivative.

FIG. 11 is a flow chart illustrating an exemplary discovery strategy.

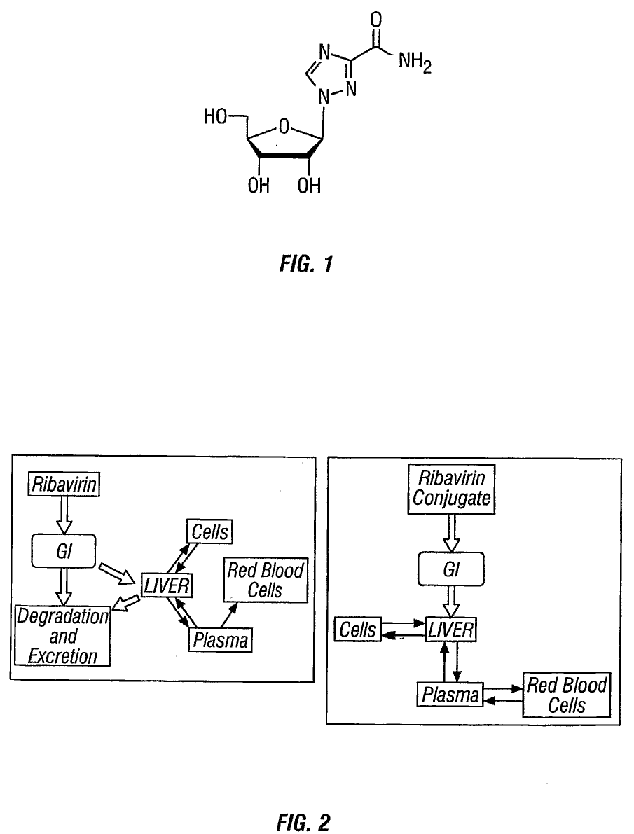

### DETAILED DESCRIPTION OF THE INVENTION

Throughout this application the use of “peptide” is meant to include a single amino acid, a dipeptide, a tripeptide, an oligopeptide, or a polypeptide. Throughout this application the use of “glycopeptide” is meant to include a carbohydrate covalently attached to a single amino acid, a dipeptide, a tripeptide, an oligopeptide, or a polypeptide. Throughout this application the use of “carrier peptide” is meant to refer to the peptide or glycopeptide. Throughout this application the use of “prodrug” and/or “derivative” is meant to refer to a peptide ribavirin conjugate and/or a glycopeptide ribavirin conjugate.

At times the invention is described as being a ribavirin attached to a peptide or glycopeptide to illustrate specific embodiments of the ribavirin conjugate. Preferred lengths of the conjugates and other preferred embodiments are described herein.

Modulation is meant to include at least the affecting of change, or otherwise changing total absorption, rate of adsorption and/or target delivery. Sustained release is at least meant to include an increase in the amount of reference drug in the blood stream for a period up to 36 hours following delivery of the carrier peptide ribavirin composition as compared to the reference drug delivered alone. Sustained release may further be defined as release of the ribavirin into systemic blood circulation over a prolonged period of time relative to the release of the ribavirin in conventional formulations through similar delivery routes.

The ribavirin may be released from the composition by enzyme-catalysis or it may be released by a pH-dependent chemical catalysis. In a preferred embodiment, the ribavirin is released from the composition by enzyme-catalysis. In one embodiment the ribavirin is released from the composition in a sustained release manner. In another embodiment, the sustained release of the ribavirin from the composition has zero order, or nearly zero order, pharmacokinetics.

The present invention provides several benefits for ribavirin delivery. While most of the current ribavirin therapeutic agents still require either IV or intramuscular (IM) injections, the present invention provides ribavirin conjugates that can be delivered much less invasively. The stability of such peptide prodrugs not only depends on amino acid sequence and linkage, but also on the type of the amino acids. Most natural amino acids have an L-configuration. However, incorporation of D-amino acids, synthetic amino acids, and N-methyl amino acids may significantly increase the metabolic stability of peptides and their derivatives. In combination with branched peptide linkages (the drug is attached to the side-chain of an amino acid), these non-natural peptide conjugates are less likely to be substrates for digestive or blood enzymes while still being recognized by less specific liver enzymes.

Selection of the amino acids will depend on the physical properties desired. For instance, if an increase in bulk or lipophilicity is desired, then the carrier peptide will be enriched in the amino acids that have bulky, lipophilic side chains. Polar amino acids, on the other hand, can be selected to increase the hydrophilicity of the peptide. Ionizing amino acids can be selected for pH controlled peptide unfolding. Aspartic acid, glutamic acid, and tyrosine carry a neutral charge in the stomach, but will ionize upon entry into the intestine. Conversely, basic amino acids, such as histidine, lysine, and arginine, ionize in the stomach and are neutral in an alkaline environment.

Other factors such as π-π interactions between aromatic residues, kinking of the peptide chain by addition of proline, disulfide crosslinking, and hydrogen bonding can all be used to select the optimum amino acid sequence for a desired performance parameter. Ordering of the linear sequence can influence how these interactions can be maximized and is important in directing the secondary and tertiary structures of the polypeptide.

Variable molecular weights of the carrier peptide can have profound effects on the ribavirin release kinetics. As a result, low molecular weight ribavirin delivery systems are preferred. An advantage of this invention is that chain length and molecular weight of the peptide can be optimized depending on the level of protection desired. This property can be optimized in concert with the kinetics of the first phase of the release mechanism. Thus, another advantage of this invention is that prolonged release time can be imparted by increasing the molecular weight of the carrier peptide.

In one embodiment the ribavirin is attached to a peptide that ranges between a single amino acid and 450 amino acids in length. In another embodiment, two to 50 amino acids are preferred, with the range of one to 12 amino acids being more preferred, and one to 8 amino acids being most preferred. In another embodiment, the number of amino acids is selected from 1, 2, 3, 4, 5, 6, or 7 amino acids. In another embodiment, of the invention the molecular weight of the carrier portion of the conjugate is below about 2,500, more preferably below about 1,000 and most preferably below about 500.

Optimally, the chain length of the peptide should be as short as possible to minimize cost and time for development and production. Preferably, conjugates should have chain lengths ranging from two to a maximum of five amino acids. Peptides with more than five amino acids may not survive GI digestion and are expensive to manufacture. Dipeptide and tripeptide derivatives are especially preferred.

The glycopeptides of the invention contain at least one carbohydrate moiety which is linked to the peptide chain. Possible carbohydrate candidates include galactose, mannose, and their analogs. These two sugars express the best affinities for hepatic asialo glycoprotein receptors.

Compositions of the invention comprise three essential types of attachment. These types of attachment are termed: C-capped, N-capped and side-chain attached. C-capped comprises the covalent attachment of a ribavirin to the C-terminus of a peptide either directly or through a linker. N-capped comprises the covalent attachment of a ribavirin to the N-terminus of a peptide either directly or through a linker. Side-chain attachment comprises the covalent attachment of a ribavirin to the functional side chain of a peptide either directly or through a linker. Amino acids with reactive side chains (e.g., glutamic acid, lysine, aspartic acid, serine, threonine and cysteine) can be incorporated for attaching multiple ribavirins or adjuvants to the same carrier peptide. The present invention also envisions the use of multiple ribavirin moieties along a carrier chain.

There are four sites on ribavirin where the C-terminus of a peptide can be attached: 2′-—OH, 3′—OH, 5′—OH, and nucleobase amide function.

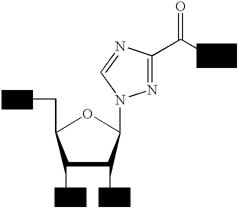

In one embodiment of the invention, the preferred substituted derivative to be modified is at the 5′-position of the carbohydrate ring. This primary alcohol is the least sterically hindered position on the carbohydrate ring. Attachment to any of the other hydroxyl groups is also possible, but synthetically more challenging. While the two secondary hydroxyl groups can easily be protected with an isopropylidene group, selective protection of 3′- and 5′—OH, or 2′- and 5′—OH requires a more complex pathway. Moreover, direct blocking of the 5′—OH appears to be the best way of avoiding phosphorylation and thus accumulation in RBCs. Similarly, peptide addition to the amide group of the base moiety leaves an unprotected 5′—OH and furthermore, may result in an undesired conversion to the respective carboxylic acid derivative of the parent drug during eventual peptide cleavage. In addition, peptides may be added to more than one functional group of the nucleoside to create a multi-substituted derivative.

There are primarily two ways of coupling a peptide to a sugar hydroxyl group. The first is attachment of the peptide C-terminus via an ester linkage. Another approach involves conjugation of the nucleoside to the side-chain function of a suitable amino acid (e.g., Asp, Glu, Lys, Ser, Thr, Tyr) of the peptide. These linkages could be ester, amide, carbamate, carbonate, or ether bonds. Since the connection between peptide and nucleoside determines the stability of the conjugate, this variety of different coupling options will increase the chances of finding suitable lead compounds that will demonstrate the desired properties.

Similarly, glycopeptide conjugates can be synthesized by adding a carbohydrate moiety to the N-terminus or to a side-chain function of an already coupled peptide. A straightforward way of attaching a sugar to a peptide is via a carbamate bond (FIG. 8). If these glycopeptides express insufficient stability, other linkage types can be explored, e.g., carbonate or ether bonds.

The present invention now will be explained with reference to the following non-limiting examples.

### EXAMPLES

### Example 1:

### Preparation of Peptide/Glycopeptide Derivative of Ribavirin

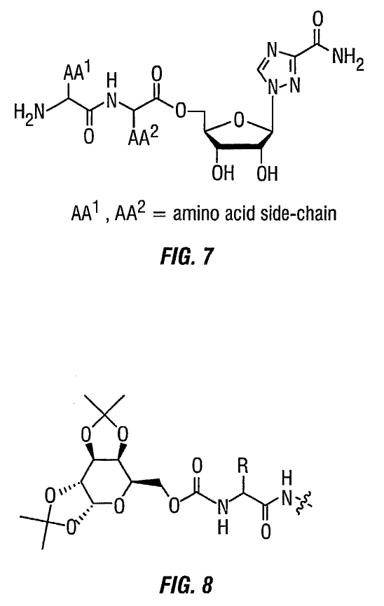

We have prepared a number of peptide and/or glycopeptide derivatives of ribavirin (Table 2) to explore synthetic feasibility and preliminary screening methods of such compounds (FIG. 7). We found that these conjugates can be readily obtained using standard peptide and nucleoside chemistries.

TABLE 2
Peptide/glycopeptide derivatives of ribavirin synthesized.
Current Compound Library

Ala-Ile-Rib
Ala-Pro-Rib
Asp-Asp-Rib
Asp-Asp-Rib
D-Lys-Lys-Rib
D-Phe-Pro-Rib
Gal-Gly-Gly-Rib
Gal-Pro-Phe-Rib
Glu-Glu-Rib
Gly-Gly-Rib
Gly-Leu-Rib
Leu-Leu-Rib
Leu-Phe-Rib
Leu-Pro-Rib
Lys-Lys-Rib
Phe-Ala-Rib
Phe-Gly-Rib
Phe-Leu-Rib
Phe-Phe-Rib
Phe-Pro-Rib
Phe-Rib
Pro-Ile-Rib
Pro-Phe-Rib
Pro-Pro-Rib
Val-Pro-Rib
Val-Val-Rib

Some of these conjugates were tested for their stability to GI digestion using in vitro assays with enzymes typically found in the GI tract: pepsin, esterase, and pancreatin. The obtained data showed that most ribavirin dipeptides released only minimal amounts of active drug under the given conditions.

### Experimental Design and Methods

The present invention relates to the discovery of a prodrug of ribavirin that, when administered orally, is absorbed intact into the bloodstream, circulated, and metabolized hepatically into ribavirin. This discovery has involved the synthesis of small peptide and glycopeptide prodrugs of ribavirin and the characterization of their stability, absorption, and metabolism in various in vitro assays. As detailed below, the synthetic procedure for preparing esters of ribavirin follows standard solution-phase peptide and carbohydrate chemistries, both of which are well established in the art. Furthermore, the in vitro assays to evaluate stability, absorption, and metabolism are well documented and represent models of gastrointestinal digestion, intestinal absorption, plasma half-life, whole blood disposition, and hepatic uptake and metabolism. Selected ribavirin conjugates that meet some or all of the proposed criteria in vitro have been tested for their pharmacokinetic profile and distribution in vivo.

### Synthetic Strategy

The methods of covalently attaching peptide chains via an ester linkage to a nucleoside, such as ribavirin, involve standard peptide and carbohydrate chemistry. The process begins with the synthesis of peptide precursors or direct coupling of single amino acids to the drug of interest. The amino acid or peptide side-chain can then be extended by condensating an additional peptide (usually a dipeptide) succinimide ester with the intermediate. Depending on the amino acids and the free drug, we found that the initial coupling reactions can be performed most effectively using one of the following two techniques: 1) addition of amino acid/peptide succinimide esters or 2) direct coupling with activating agents (e.g., HBTU, TSTU). In the case of ribavirin, the succinimide ester method provide positive results with respect to reaction time, yield, and purity.

### Synthetic Scheme

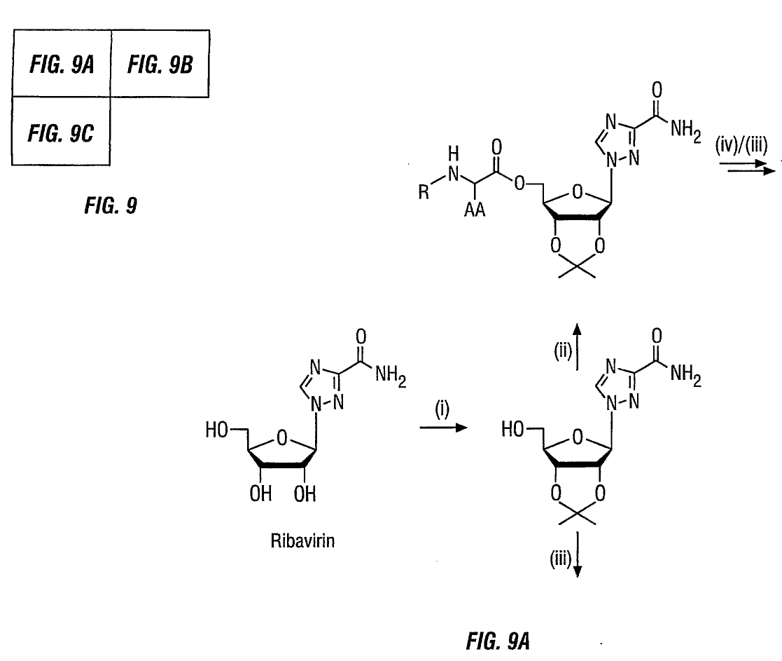

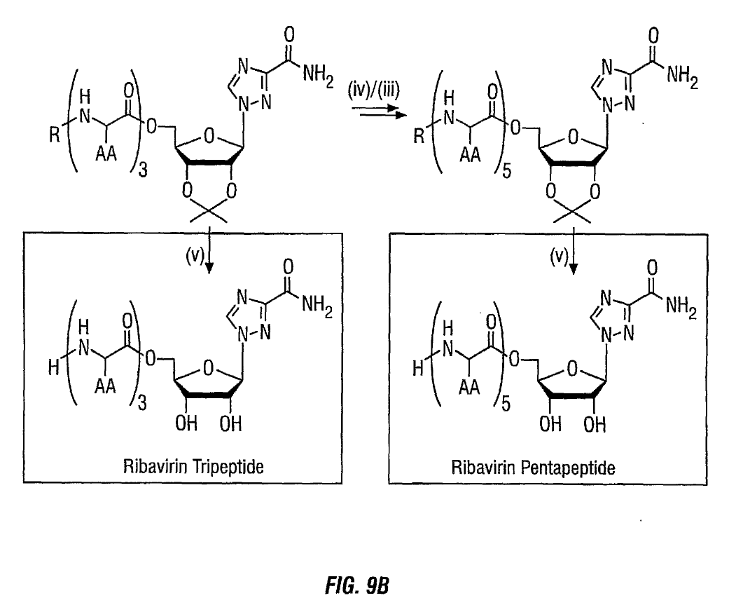

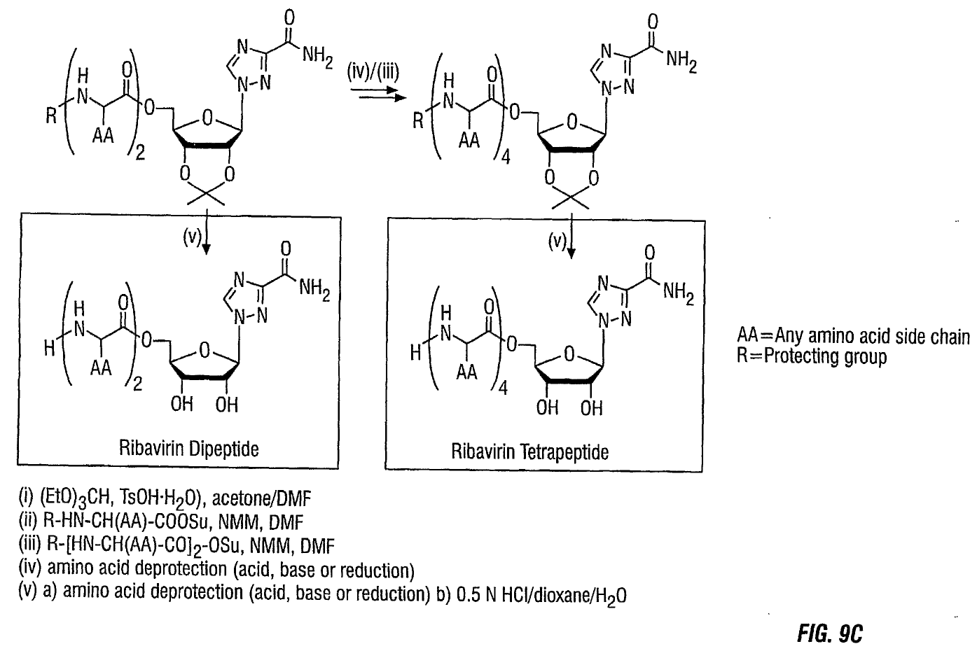

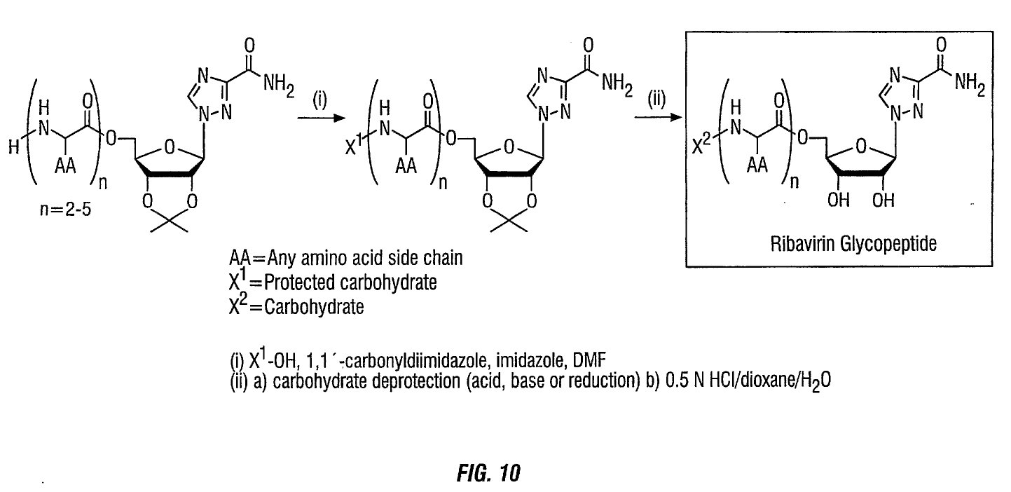

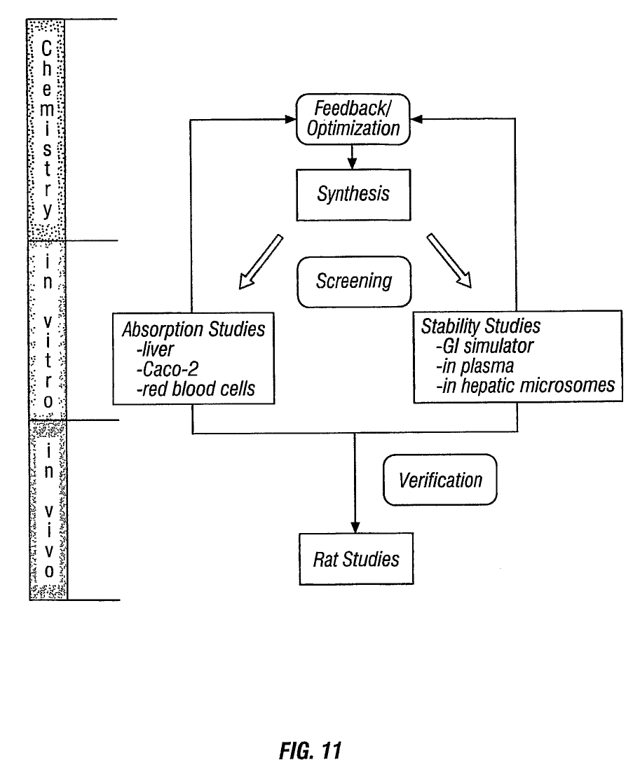

We have developed a synthetic pathway that has allowed us to successfully synthesize the compounds listed in Table 2 (FIGS. 8 and 9). The total synthesis is short (3-7 steps) allowing us to explore the maximum number of variables in a brief period of time and to respond quickly to screening results (FIG. 8). In the first step ribavirin was protected with an isopropylidene group. Subsequent coupling with an N-protected dipeptide yielded the precursor for the final peptide or glycopeptide derivative. The former was readily obtained after a single deprotection reaction. Preparation of the latter called for selective deprotection of the N-terminus of the peptide without cleaving the isopropylidene group or the protection of a potential amino acid with side-chain function (e.g., Asp, Glu, Lys). The resulting intermediate was coupled with a sugar derivative and then completely deprotected to give the final ribavirin glycopeptide (FIG. 9).

Preparation of Ribavirin-2′,3′-isopropylidene

Anhydrous triethylorthoformate (2.2 eq) and toluenesulfonic acid monohydrate (0.022 eq) were dissolved in anhydrous acetone. The solution was stirred for 20 h at ambient temperature. Ribavirin (1 eq) was dissolved in as little anhydrous N,N-dimethylformamide as possible and subsequently added to the acetone solution. The mixture was heated for 20 h to 50° C. Solvents were evaporated to dryness. The remaining residue was purified via column chromatography (0-8% MeOH/CHCl₃).

Preparation of Protected Ribavirin Dipeptide Derivative

Isopropylidene protected ribavirin was dissolved in anhydrous N,N-dimethylformamide. N-Methylmorpholine (5 eq) and protected dipeptide succinimide ester (1 eq) were added. The solution was stirred for 20 h at ambient temperature. Solvents were evaporated and saturated sodium bicarbonate solution was added to the remaining residue. The suspension was stirred for 45 min. Solid crude product was filtered off and purified via HPLC.

Selective Deprotection of Boc-protected Peptide/Amino Acid Side Chain

Protected conjugate was dissolved in anhydrous 1,2-dioxane. Subsequently, 4 N hydrochloric acid in 1,2-dioxane solution was added to produce a total of 2 N hydrochloric acid in the mixture. The suspension was stirred for 3 h at ambient temperature. Solvents were evaporated to dryness yielding product with satisfactory purity.

Deprotection of Boc-protected Peptide/Amino Acid Side Chain and Ribavirin

Peptide/amino acid side chain and ribavirin protected conjugate was dissolved in anhydrous 1,2-dioxane. The solution was acidified with 4 N hydrochloric acid in 1,2-dioxane until the total concentration of hydrochloric acid reached 2 N. The mixture was stirred for 3 h at ambient temperature and was then diluted with water to a total hydrochloric acid concentration of 0.5 N. The solution was stirred again for 3 h at ambient temperature. Solvents were evaporated to dryness resulting in product with satisfactory purity.

Coupling Between the Free N-terminus of a Ribavirin Peptide and the Hydroxyl Group of a Carbohydrate Derivative (e.g., 1,2:3,4-Di-O-isopropylidene-α-D-galactopyranose)

The carbohydrate derivative (1 eq) was dissolved in anhydrous N,N-dimethylformamide. To the solution was added 1,1′-carbonyldiimidazole (CDI; 1.1 eq) and the mixture was stirred for 2 h at ambient temperature. The ribavirin peptide conjugate (1.5 eq) and imidazole (0.2 eq) were dissolved in anhydrous N,N-dimethylformamide. This solution was added to the activated carbohydrate and the resulting mixture was heated for 24 h to 55° C. Solvents were evaporated and the remaining residue was purified via column chromatography (0-3% MeOH/CHCl₃).

### In Vitro Screening Assays

Analysis of Free and Conjugated Ribavirin

Free ribavirin and its peptide conjugates will be analyzed by LC-MS. The LC-MS system will consist of an Agilent 1100 series binary pump, vacuum degasser, autosampler, thermostatted column compartment, variable wavelength detector, and the MSD SL quadrupole mass spectrometer equipped with an electrospray ionization source. Separation will be achieved with a 150×4.6 mm PrincetonSPHER-100 amino column (Princeton Chromatography, Princeton, N.J.) maintained at 30° C. using an isocratic mobile phase consisting of 80% MeCN and 20% NH₄OAc (10 mM, pH 3.5) and a flow rate of 1 ml/min. The UV detector will be set to a wavelength of 210 nm. MS conditions, including fragmentor, capillary voltages and spray chamber parameters, are currently being optimized for maximum sensitivity of free ribavirin and its conjugated analogs.

Unless indicated otherwise, after incubating each assay for the indicated time, preparation of samples for analysis will follow a twofold dilution with MeCN containing 1% H₃PO₄ to induce precipitation of insoluble material. Precipitant will be spun down and supernatant filtered through a Teflon filter (0.2 μm) into HPLC vials for analysis. Conjugates will be analyzed in triplicate for the presence of both free ribavirin and its parent conjugate.

Preparation of Ribavirin and Conjugate Stock Solutions

Stock solutions of ribavirin conjugates will be prepared prior to analysis and kept at −80° C. until needed for assay. To avoid excessive freeze-thaw cycles of stock solutions, small aliquots will be stored separately and diluted when necessary for analysis. Stocks will be prepared at 100-fold of the targeted in-assay concentration of each conjugate and, as noted below, either diluted within the assay or immediately prior. If needed, organic solvents (e.g., MeOH, MeCN) will be kept at concentrations below 50% in these stock solutions in order to keep in-assay concentrations below 1%. The projected concentration of all conjugates within each assay will be 10 μM depending on analytical sensitivity. Stock solutions of free ribavirin will be prepared freshly at the time of each assay and calibration standards of each conjugate will be diluted to their respective in-assay concentrations.

Stability of Ribavirin Conjugates to Proteolytic Digestion

Several in vitro enzyme assays have been developed to determine if ribavirin can be released from its prodrug conjugate. To assess whether enzymatic digestion occurs in a model of the stomach or the upper intestinal tract, USP protocols for gastric and intestinal simulation will be followed with slight modifications. U.S. Pharmacopeia and National Formulary (2000) Reagents, Indicators, and Solutions p. 2235-2236. Assays with esterase isolated from porcine liver will be used as a model for hepatic digestion of ribavirin conjugates. Furthermore, a non-specific protease isolated from Streptomyces griseus (type XIV, pronase) will be used to determine general hydrolyzability of conjugated ribavirin.

Prior to use, each enzyme stock solution will be prepared at twofold assay concentration as follows. For the gastric simulator, pepsin purified from porcine stomach mucosa will be added to a NaCl buffer (68 mM, pH 1.2) at a concentration of 6.4 mg/ml. A solution of pancreatin isolated from porcine pancreas will be prepared at a concentration of 20 mg/ml in a KH₂PO₄ buffer (100 mM, pH 7.5) for the intestinal simulator. Intestinal simulator buffer will also be used for digestions with pronase (6 mg/ml).

For esterase activity experiments, an enzyme stock solution will be prepared at a concentration of 6.6 mg/ml in 50 mM Na₂HPO₄ buffer (pH 8). Stocks of ribavirin conjugates will be diluted 50-fold in water before being diluted twofold with the enzyme (1 ml final volume) to initiate each assay. Samples will then be incubated for 1 hour (pepsin, pronase, esterase) and 4 hours (pancreatin) at 37° C. in a vortex incubator. The release of tyrosine from casein (pancreatin, pronase) and hemoglobin (pepsin) as well as 4-nitrophenol from 4-nitrophenyl acetate (esterase) will be used as controls for enzyme activity.

Caco-2 Model for Intestinal Absorption

Caco-2 cells derived from a human colonic adenocarcinoma possess many of the properties of the small intestine. They represent a useful and well-accepted in vitro model used to predict the absorption of drugs across the intestinal mucosa. Caco-2 cells plated on a membrane support allow the study of drug transport from the apical (intestinal lumen) to the basolateral (blood) side of the gastrointestinal tract. Pre-plated Caco-2 cells will be purchased from In Vitro Technologies (IVT, Baltimore, Md.) to determine absorption and permeability (and possible metabolism) of conjugated ribavirin. Prior to shipment, all pre-plated Caco-2 monolayers meet a stringent set of quality control criteria including, among others, transepithelial electrical resistance, an assessment of monolayer integrity. A technical protocol available from IVT will be followed for the determination of apical to basolateral drug transport. In Vitro Technologies, Inc., Instructions for Using Plated Caco-2 Cells: Transport Study, Apical to Basolateral (2003).

Upon receipt of Caco-2 kits, cells will be incubated in shipping media in a CO₂ incubator (5% CO₂, 37° C.) for 24 hours. Media used during apical to basolateral Caco-2 transport assays will be prepared from supplied Transport Media. Basolateral Transport Media (BTM) will be prepared by adjusting its pH to 7.4 with 1 N NaOH, while Apical Transport Media (ATM) will be prepared by adjusting its pH to 6.5 with 1 N HCl. After adding 0.6 ml of BTM to the basolateral well, each transwell containing Caco-2 cells will be carefully “rinsed” with ATM, drained, and inserted into its well. Next, 0.1 ml of dosing solution in ATM will be gently added to the apical side of each transwell and the system will be incubated (5% CO₂, 37° C.) for 1 hour. Basolateral media will then be removed from each well, filtered into HPLC vials and analyzed by LC-MS as indicated.

Plasma Stability and Whole Blood Disposition

Ribavirin accumulates in RBCs possibly causing hemolytic anemia during HCV therapy. As designed in this proposal, 5′-conjugated ribavirin prodrugs that are stable to hydrolysis in plasma cannot be phosphorylated and thus should be in cell diffusion equilibrium without accruing in RBC (if they enter RBCs at all). Moreover, plasma stability is critical in order to have conjugates reach the liver intact. Thus, both the plasma stability and the uptake of ribavirin conjugates into RBCs will be evaluated.

A previously established protocol will be followed for determining the stability of ribavirin conjugates in human plasma. Aggarwal, S. K., et al., J. Med Chem. 1990, 33,1505-1510. Conjugates (from 100-fold stock) or free ribavirin solutions will be diluted directly in plasma (e.g., 10 μl conjugate solution with 990 μl plasma) before incubation at 37° C. After 1 hour, samples will be prepared for analysis as specified.

Depending upon their stability in plasma, selected ribavirin conjugates will be assessed for their uptake into RBCs. Applying a slightly modified existing protocol, Homma, M., et al., Antimicrob. Agents Chemother. 1999, 43(11), 2716-2719, both free ribavirin and conjugates will be diluted 100-fold in whole blood collected into heparinized tubes from rats (rattus norvegicus). After incubation with gentle shaking for 1 hour at 37° C., samples will be centrifuged (1500 G, 15 min.) to separate plasma and RBCs. Plasma will be removed and prepared for analysis as indicated above. To determine ribavirin and conjugate uptake into RBCs, remaining cells will be lysed and treated with acid phosphatase prior to preparation for analysis.

Hepatic Absorption and Metabolism

Isolated human liver microsomes and hepatocytes exhibit many of the features of the intact liver and are widely accepted models for investigating drug metabolism. Human hepatocytes express many typical hepatic functions and express metabolic enzymes providing the closest in vitro model to human liver. Based on this model, similarities between in vitro and in vivo metabolism of drugs have been observed. Gomez-Lechon, M. J., et al., Curr. Drug Metab. 2003, 4(4), 292-312. Thus, human liver microsomes and pre-plated human hepatocytes will be purchased from In Vitro Technologies (IVT, Baltimore, Md.) and used to determine hepatic absorption and metabolism of conjugated ribavirin.

Conversion of conjugates into ribavirin will be monitored in human hepatic microsomes. The stability of each conjugate will be determined by following a technical protocol available from In Vitro Technologies. In Vitro Technologies, Inc., Instructions for Using Microsomes and S9 fractions, 2003. The general procedure is as follows: a 2% (w/v) NaHCO₃ buffer containing 1.7 mg/ml NADP, 7.8 mg/ml glucose-6-phosphate, and 6 units/ml glucose-6-phosphate dehydrogenase (G6PD) will be prepared. This activation buffer will be kept at 4° C. until used and will be stable for up to 8 hours. To 16×100 mm test tubes, 50 μl of microsomes (from 2 mg/ml stock), 10 μl of conjugate (from 100-fold stock), and 690 μl of 2% NaHCO₃ buffer (not activation buffer) solution will be added. The mixture will be vortexed gently for 5-10 min. at 37° C. followed by the addition of 250 μl of activation buffer (1 ml final volume). Samples will be incubated for 1 hour at 37° C. before preparation for analysis.

Assay conditions for monitoring the absorption and metabolism of ribavirin conjugates in plated hepatocytes, are described in a technical protocol available from IVT. In Vitro Technologies, Inc., Cultured Hepatocyte Xenobiotic Metabolism Assay, 2001. Prior to their addition to cells, conjugate stock solutions will be diluted 100-fold to in-assay concentrations in Hepatocyte Incubation Media (HIM) supplied with each kit. Existing media will be removed from cells and replaced with media containing 10 μM of conjugate. Treated cells will be incubated in a CO₂ incubator (5% CO₂, 37° C.) for 1 hour. After incubation, media will be removed and prepared for analysis to determine levels of unabsorbed conjugate. The cells will then be washed (with HIM) and aspirated twice. Remaining cells will be extracted from each well with two volumes (of initial well volume) of MeCN (1% H₃PO₄) and prepared for analysis to determine the absorption of conjugate and its conversion to free ribavirin.

### In Vivo Models

Early on in prodrug discovery, it is vital to identify the significance of in vitro data. We have found that, typically, the use of in vitro models leaves out many of the actual biological processes that occur during the lifecycle of a prodrug. This can include but is not limited to digestive enzymes or bile salts, mechanical action, active transport mechanisms, passive diffusion mechanisms, bacterial degradation/metabolism, alternative metabolic pathways and clearance mechanisms. However, all of these factors can be considered when the prodrug is administered to an animal. That is why we believe that it is important to identify the relevance of each in vitro assay and its correlations to in vivo animal models.

In the interest of animal welfare and discovery throughput, we have decided to develop an extensive library of in vitro assays using already proven technologies and then use the data from these assays to categorize our first generation of compounds. Several of the top performing compounds (nine prodrugs with one reference standard) will be administered orally to rats whose blood and livers will then be analyzed. This initial data will help us to evaluate the relevance of our in vitro models and to decide whether we should continue its use. Should the in vitro models fail to correlate with in vivo data, we would then need to rely more heavily on animal models.

After the significance of the in vitro models is established, we then would only use in vivo models to help verify the pharmacokinetics of the prodrugs and to decrease the number of potential lead compounds (four prodrugs with one reference standard).

### Discovery Strategy

When designing and testing prodrugs the number of variables should initially be minimized while the information obtained from these compounds should be maximized. Our approach to discovery has been extremely successful in past projects and is shown in FIG. 9. This same approach will be implemented for the research on ribavirin conjugates. Initially, our first generation compounds will be screened to establish the in vitro/in vivo correlation described above. Using this data, we can then optimize the desired properties using the continuous flow of in vitro data to optimize the next set of compounds.

For example, if data suggests that attachment to the 5′-position reduces accumulation in RBCs, all future conjugates would focus on this trend. If a particular chain length has the desired properties, then the spotlight could be shifted to develop only those conjugates with that chain length. The feedback from the in vitro/in vivo data would continually generate better sets of ribavirin conjugates.

### Example 2

### Peptide Conjugates Surviving Digestion and Intestinal Absorption

By adding an amino acid or small peptide to a drug, not only can bioavailability be increased by utilizing both active and passive transport mechanisms, but significant levels of intact prodrug can reach the blood. A classic example of this method is the L-valine prodrug of acyclovir (valacyclovir). The nucleoside mimic acyclovir is an anti-viral drug with poor bioavailability (15-30%) that once attached to valine via an ester bond, increases its bioavailability twofold (54%). Most studies suggest that the amino acid prodrug conjugate is found in the serum and actively transported through a dipeptide transporter. Once absorbed, valacyclovir likely undergoes intestinal and hepatic hydrolysis to L-valine and acyclovir.

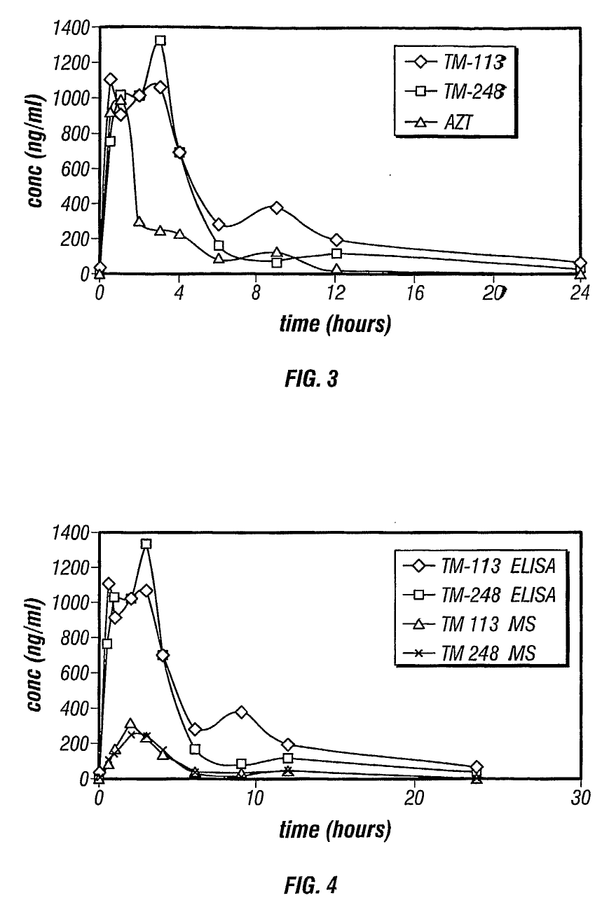

Other examples of improved absorption combined with considerable conjugate levels of an antiviral in the serum were produced. AZT, a nucleoside analog used in AIDS therapy, was covalently attached to the side-chain of polyglutamic acid via an ester linkage to form Poly(Glu)-AZT. This material was then given orally to rats at equimolar amounts of AZT. Detection of AZT by ELISA demonstrated that high levels of either free AZT or partially digested Poly(Glu)-AZT (Glu(AZT)ₙ with n≧1, n=number of glutamic acid subunits) were present in the plasma of rats after oral administration (FIG. 3). Later animal studies using mass spectroscopy confirmed that ELISA was detecting mostly the conjugates, Glu(AZT)ₙ, with only low serum levels of free AZT (FIG. 4).

A test series with numerous AZT conjugates showed that the typical ELISA response for these prodrugs is always lower than or equal to the response for free AZT. It thus appears that the large difference in concentrations between the ELISA and LC-MS/MS (which only detects free AZT) plots is caused by a considerable amount of Glu(AZT)n that survives digestion and absorption intact.

### Example 3

### Peptide Conjugates Stable in Blood

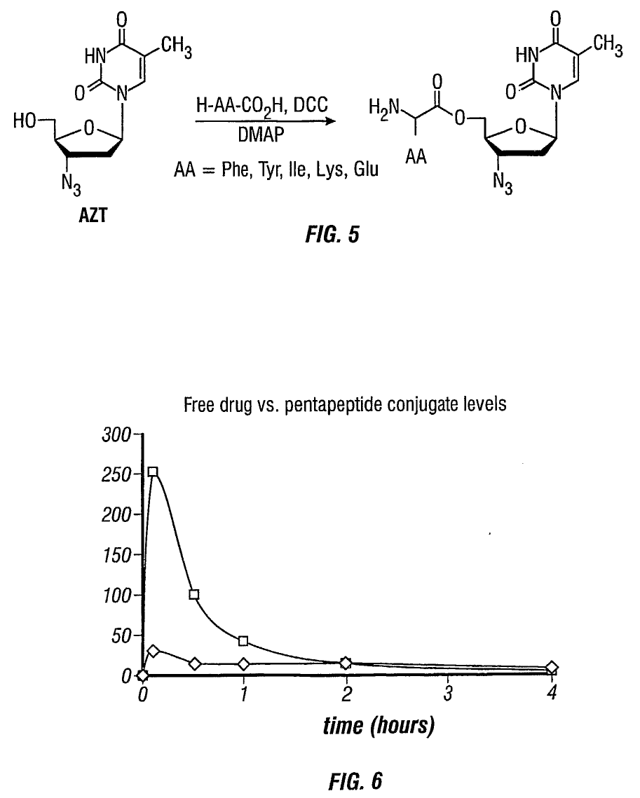

In the literature, there are several cases of amino acid prodrugs that survive intact within the blood. In one example, the anti-HIV agent AZT was coupled to the C-terminus of several amino acids via an ester linkage (FIG. 5). These conjugates were then incubated in human plasma and rat hepatic microsomes. Aggarwal, S. K., et al., J. Med. Chem. 1990, 33, 1505-1510. The hydrolysis half-life (t_{1/2}) of these compounds in human plasma ranged from 20 min. for the phenylalanine derivative to greater than 240 min. for the isoleucine derivative (Table 1). In rat hepatic microsomes, the t_{2/2} ranged from 5 min. for tyrosine and 30 min. for glutamic acid and phenylalanine.

TABLE 1
Hydrolysis t_{½} of certain AZT prodrugs.

| 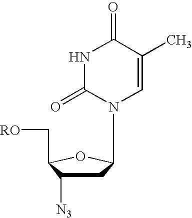 R | in vitrohydrolysis t_{½} [min.]human plasma rat hepatic microsomes | |
|---|---|---|
| Phe | 20 | 30 |
| Tyr | 60 | 5 |
| Ile | >240 | 19 |
| Lys | 30 | 14 |
| Glu | 70 | 30 |

We have discovered several instances of ester prodrugs that survive intact in the blood. One example was a drug that was conjugated via an ester bond to the C-terminus of a pentapeptide. When administered intravenously (IV), this conjugate had a small amount of drug release initially but up to 85% survived intact through four hours (FIG. 6). This data suggests that serum enzymes do not cleave most of this prodrug before it clears the circulating system.

### Example 4

### Selective Drug Delivery

An example of a prodrug demonstrating drug targeting attributes was ribavirin connected to lactosaminated poly-L-lysine (L-Poly(Lys)-RIBV). Fiume, L., et al., J. Vir. Hep. 1997, 4(6), 363-370. This conjugate inhibited murine hepatitis virus (MHV) replication when injected intramuscularly into mice. The compound was selectively absorbed by the liver ([³H]-labeled ribavirin showed a 4.7:1 ratio of dpm in liver vs. dpm in RBC) and did not release any drug when incubated with human or mouse blood.

Based on these experiments, we theorize that a small peptide or glycopeptide derivative of ribavirin would be the most suitable prodrug for combination therapy of chronic HCV for the following reasons: conjugates containing macromolecules such as human albumin exhibit poor solubility and stability in the gastrointestinal (GI) tract, and as a consequence require IV administration; similarly, lactosaminated L-Poly(Lys)-RIBV calls for IM injection and consists of an ambiguous polymer mixture of individual components with various sites of drug attachment; ribavirin precursors like viramidine necessitate conversion of one nucleoside analog into another to exert activity. Wu, J. Z., et al., J. Antimicrob. Chemother. 2003, 52, 543-546. The precursor itself may exhibit additional toxicity and, without selective delivery, does not eliminate the toxic effects of ribavirin after being metabolized. Our prodrugs, however, will decrease side effects through liver targeting and by not altering the actual active drug moiety will unlikely display previously unobserved toxicities.

It will be understood that the specific embodiments of the invention shown and described herein are exemplary only. Numerous variations, changes, substitutions and equivalents will occur to those skilled in the art without departing from the spirit and scope of the invention. In particular, the terms used in this application should be read broadly in light of similar terms used in the related applications. Further, it should be recognized that it is within the skill of one in the art to use various features from one described embodiment with features from another embodiment. Accordingly, it is intended that all subject matter described herein and shown in the accompanying drawings be regarded as illustrative only and not in a limiting sense and that the scope of the invention be solely determined by the appended claims.

1. A compound having the formula: 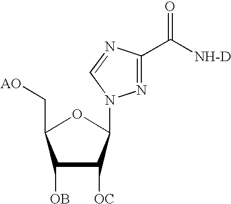
wherein each of A, B, C and D is independently an amino acid, a peptide, a glycopeptide or hydrogen; with the proviso that A, B, C and D are not all simultaneously hydrogen H.

2-3. (canceled)

4. The compound of claim 1, wherein A is a peptide selected from a dipeptide, a tripeptide, a tetrapeptide and a pentapeptide.

5-6. (canceled)

7. The compound of claim 1, wherein at least one of A, B, C and D is a glycopeptide in which the carbohydrate is attached to the N-terminus of the peptide.

8. The compound of claim 1, wherein at least one of A, B, C and D is a glycopeptide in which the carbohydrate is attached to a side-chain of the peptide.

9. The compound of claim 1, wherein A is a peptide selected from Ala-Ile-, Ala-Pro-, Asp-Asp-, D-Lys-Lys-, D-Phe-Pro-, Gal-Gly-Gly-, Gal-Pro-Phe-, Glu-Glu-, Gly-Gly-, Gly-Leu-, Leu-Leu-, Leu-Phe-, Leu-Pro-, Lys-Lys-, Phe-Ala-, Phe-Gly-, Phe-Leu-, Phe-Phe-, Phe-Pro-, Phe-, Pro-Ile-, Pro-Phe-, Pro-Pro-, Val-Pro-, and Val-Val-.

10. The compound of claim 1, wherein the compound exhibits lower toxicity relative to the compound wherein A, B and C are hydrogen atoms.

11. (canceled)

12. The compound of claim 1, wherein the compound is stable during the digestion process, survives absorption into circulation, and reaches the liver intact.

13. The compound of claim 1, wherein the total average bioavailability in a human is greater than 64 percent.

14. (canceled)

15. A method of treating a viral infection in a patient comprising administering to said patient a therapeutically effective amount of a compound having the formula: 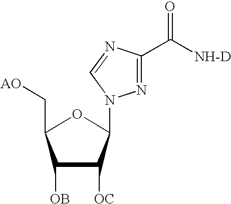
wherein each of A, B, C and D is independently an amino acid, a peptide, a glycopeptide or hydrogen; with the proviso that A, B, C and D are not all simultaneously hydrogen.

16. The method of claim 15, wherein the compound is administered orally.

17. The method of claim 15, wherein said viral infection is an infection of hepatitis C virus, infant respiratory syncytial virus, influenza A virus, influenza B virus, hepatitis A virus, hepatitis B virus, Lassa fever virus, Hantaan virus, or the respiratory virus that causes SARS.

18. The method of claim 17, wherein the patient has been infected with hepatitis C virus.

19. A pharmaceutical composition comprising a compound having the formula: 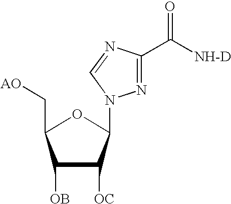
wherein each of A, B, C and D is independently an amino acid, a peptide, a glycopeptide or hydrogen; with the proviso that A, B, C and D are not all simultaneously hydrogen, and a pharmaceutically acceptable excipient.

20. (canceled)

21. The pharmaceutical composition of claim 19 which is in a form suitable for oral administration.

22. (canceled)

23. The method of claim 15, further comprising co-administering the compound with interferon.

24. The method of claim 23, wherein the interferon is selected from interferon alpha-2a, interferon alpha-2b, peginterferon alpha-2a and peginterferon alpha-2b.

25-27. (canceled)
